(19) United States
(12) Patent Application Publication
(10) Pub. No.: US 2001/0054429 A1
(43) Pub. Date: Dec. 27, 2001

(54) Methods for collecting cord blood and related devices

(76) Inventor: Frank Robert Witter, Baltimore, MD (US)

Correspondence Address: Dike, Bronstein, Roberts & Cushman, Intellectual Property Practice Group, EDWARDS & ANGELL, LLP, 130 Water Street, Boston, MA, 02109, US

(21) Appl. No.: 09/832,393

(22) Filed: Apr. 10, 2001

Related U.S. Application Data
(60) Provisional application No. 60/195,936, filed on Apr. 10, 2000.

(51) Int. Cl. A61D 1/10; A61B 17/42; A61B 17/46; A61M 5/00; A61B 19/00

(52) U.S. Cl. 128/898; 604/187; 606/119

(57) ABSTRACT

Featured is a method for the collection of blood, more particularly blood from the umbilical cord, as well as shielding devices and device kits for use with such methods. Such methods and shielding devices advantageously decrease the risk or potential for accidental needle sticks when withdrawing blood from the umbilical cord. Such a method is particularly useful for collecting cord blood and includes the steps of positioning a body portion, from which blood is to be withdrawn, within a shield member and inserting an insertion member of a blood extraction device into the body portion, where the shield member is generally disposed between the insertion member and the user's hand. The method also can include holding the shield member in one of the user's hands and accomplishing said inserting using another of the user's hands. The method further includes stabilizing the body portion using a digit of the one hand holding the shield member.

This application claims the benefit of U.S. Provisional Application Ser. No. 60/195,936 filed Apr. 10, 2000, the teachings of which are incorporated herein by reference.

### BACKGROUND

1. Field of the Invention

The present invention generally relates to methods and devices for the collection of blood and more particularly to a method for collecting cord blood including devices or shields and device kits related thereto.

2. Background

The umbilical cord functions as a conduit between a mother and a fetus developing in the womb of the mother whereby oxygen, nutrients and waste products can pass between the mother and the fetus. Immediately after a baby is born, the umbilical cord is clamped and cut, thereby freeing the baby from the mother. Shortly thereafter, the placenta from which the umbilical cord extends is either expelled or removed from the mother.

It has become customary practice to take blood samples from the umbilical cord of a newborn child and to perform chemical and biological assays on the withdrawn blood. The assays are used to determine whether a potential mismatch exists between the blood types of the mother and child, whether the baby is subject to potential genetically transmitted diseases, bacterial diseases and viral infections, such as human immunodeficiency viruses which lead to AIDS and hepatitis B and C, whether the mother has ingested drugs which possibly could lead to problems such as addiction in the newborn child. It is generally desirable to recover the cord blood sample quickly and safely given the time and health concerns of the practitioner.

One method of sampling the cord blood is to unclamp the cord and drain the blood into a sterile open tube or container. Additionally, one can squeeze the section of the cut cord by hand to manually withdraw the blood from the cord. These methods carry a risk of contamination of the blood making it unreliable for some assays and unacceptable for long-term storage and later use. For example, if cells are to be used for transplantation, they must be free of bacterial contamination, which occurs in 48% of the cases when blood is allowed to drain from the cord into an open sterile container. (Elchalal U, Fasouliotis S J, Shtockheim D, Brautbar C, Schenker J G, Weinstein D et al. Postpartum umbilical cord blood collection for transplantation: a comparison of three methods. Am. J. Obstet. Gynecol. 2000; 182:227-232).

The umbilical cord is typically coated with various fluids, for example vaginal blood, amniotic fluid and Wharton's gel. In addition to creating the environment that can lead to contamination of the blood sample, these various fluids make the umbilical cord wet, slimy and extremely difficult to handle. Further, it is desirable to minimize contact between health care workers and such fluids.

Collection of umbilical cord blood following delivery thus is occurring more frequently in clinical practice by direct needle aspiration of the umbilical cord vessels. When the intent is to assess the newborn's condition at birth, this is done by direct needle sampling of the umbilical artery for blood gases. (Committee on Technical Bulletins of the American College of Obstetricians and Gynecologists. Umbilical artery blood acid-base analysis. No. 216; Nov. 1995) Placental blood is also collected as a useful source of allogeneic hematopoietic stem cells for bone marrow reconstruction. (Rubinstein P. Carrier C, Scardavou A, Kurtzberg J, Adamsen J, Migliacco A R et al. Outcome among 562 recipients of placental-blood transplants from unrelated donors. N. Engl. J. Med., 1998; 339:1565-1577).

Problems associated with inadvertent needle sticks are well recognized with blood withdrawal, catheter emplacement and other medical procedures utilizing needles. Significant attention is placed on needle stick problems due to the likelihood of the medical practitioner or other health care workers being exposed to a variety of blood born pathogens, including syphilis, hepatitis B, hepatitis C, and AIDS. A number of protective devices have been developed as a result of the widespread knowledge and history associated with medical needle care and their disposal problems.

Certain devices that have been developed for the collection and sampling of blood from the umbilical cord are reported in U.S. Pat. Nos. 5,674,227, 6,059,794 and 5,575,795. One of the illustrative devices is configured and arranged so the umbilical cord is trapped and squeezed between a lid and a tray so that blood is squeezed out of the cord and into a reservoir of the tray. Another of the illustrative devices consists of a container having two clamp members, where the cord extends between and is secured to these clamp members. The other illustrative device consists of a trough member having disposed at either end a clamp for clamping the umbilical cord. To draw the blood sample from the umbilical cord, a needle with syringe for example is inserted into the cord in the area between the clamps.

It thus would be desirable to provide a new device that shields the hand of the medical practitioner from a needle stick during the process of withdrawing cord blood, and methods for collecting cord blood using such a shielding device. It would be particularly desirable to provide such a shielding device and method that would be simple in construction in comparison to prior art devices. Such collection devices preferably also would be less costly than prior art devices and such methods would not involve highly detailed procedures or complex methods embodying multiple steps.

### SUMMARY OF THE INVENTION

The present invention features a method for the collection of blood, more particularly blood from the umbilical cord, as well as shielding devices and device kits for use with such methods. Such methods and shielding devices advantageously decrease the risk or potential for accidental needle sticks when withdrawing blood from the umbilical cord. Also such methods and shielding devices are less complex and simpler in construction and use as compared to any of a number of prior art devices and methods.

A method for the collection of blood, in particular cord blood, according to the present invention includes the steps of positioning a body portion, from which blood is to be withdrawn, within a shield member such that the shield member is generally disposed between the body portion and a hand of a user, and inserting an insertion member of a blood extraction device (e.g., a needle of a syringe) into the body portion such that the shield member is generally disposed between the insertion member and the user's hand. The method also can include holding the shield member in one hand of the user (e.g., the non-dominant hand) while accomplishing said inserting using another hand of the user (e.g., the dominant hand).

Such a method further includes stabilizing the body portion using a digit of the one hand holding the shield member. In further embodiments, such a method comprises providing a shield member one portion of which is configured and arranged so as to be releasably held in said one hand, the non-dominant hand, and another portion of which is configured and arranged to releasably receive the body portion.

In preferred embodiments, another portion of the shield member includes a passage extending along an axis of the shield member, in which passage is received the body portion. More particularly, the passage in cross-section is one of arcuate or polygonal. Further, the one portion is configured and arranged so as to be one of arcuate or polygonal in cross-section. In a generally preferred embodiment, the shield member is configured and arranged so as to be in the general shape of a hollow tube cut in half along its length.

The provided shield member is made of a material appropriate for a typical hospital use such as, for example, blood withdrawal, catheter emplacement and other medical procedures utilizing needles. Such materials in addition are those that are of the type that would be generally resistant to puncture by a needle under typical hospital use. Such materials include, but are not limited, to a metal or metal alloy such as stainless steel (e.g. 304 stainless steel), medical grade plastic or similar materials suitable for use in a medical setting.

In more specific embodiments, the provided shield member is reusable and sterilizable between uses. Alternatively, the shield member is disposable and thrown out after each use, thereby eliminating possibility of cross contamination from one patient to another.

Also featured is a device for blood collection, more particular collection of cord blood, including a shield member as herein above described. Further featured are device kits including a shield member as herein described alone or in combination with a blood extraction device such as a needle and syringe.

Other aspects and embodiments of the invention are discussed below.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIGS. 1A-C are various views of a blood collection shield according to the present invention;

FIGS. 1D and E are end views of a blood collection shield according to the present invention illustrating alternative geometrical shapes;

FIGS. 2A-C are perspective views illustrating the blood collection process according to the present invention;

FIG. 3 is a perspective view of an illustrative blood collection kit according to the present invention; and

FIG. 4 is a perspective view of an exemplary needle and syringe with a needle-shield safety device contemplated for use with the blood collection shield of the present invention.

### DETAILED DESCRIPTION OF THE INVENTION

As discussed above, the invention provides methods for the collection of blood, more particularly blood from the umbilical cord, as well as shielding devices and device kits for use with such methods. Preferred methods of the invention include positioning an umbilical cord within a shield member; and contacting the cord with a blood extraction device. The shield member is suitably disposed between the cord and a hand of a medical practitioner. A typical blood extraction device comprises a needle and syringe.

Referring now to the various figures of the drawing wherein like reference characters refer to like parts, there is shown in FIGS. 1A-C various views of an exemplary blood collection shield 100 according to the present invention. There also is shown in FIGS. 1D-E alternative blood collection shields 100a,b illustrating alternative geometrical shapes for such blood collection shields.

A general purpose of a blood collection shield according to the present invention is to improve safety to hospital personnel and/or medical practitioners particularly when drawing umbilical cord blood following delivery by significantly minimizing the risk of, more specifically preventing, accidental needle sticks. This minimizes the risk of transmission of blood born pathogens to hospital personnel/practitioners due to such accidental needle sticks at the time of blood drawing. Such a shield advantageously maintains the capability of collecting newborn cord blood for a variety of purposes from the umbilical cord and/or placenta after the baby has been delivered and the cord clamped. This allows blood sampling, that reflects the infant's status at birth without requiring actual blood drawing from the infant, and minimizes the risk of transmission of pathogens to hospital personnel or medical practitioners drawing the blood sample(s).

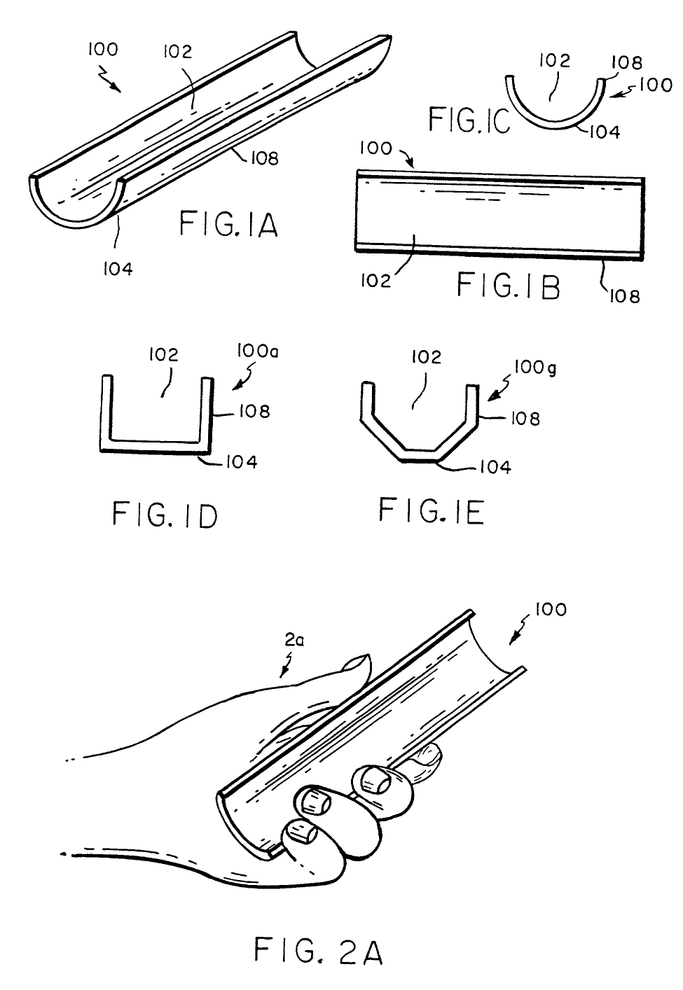

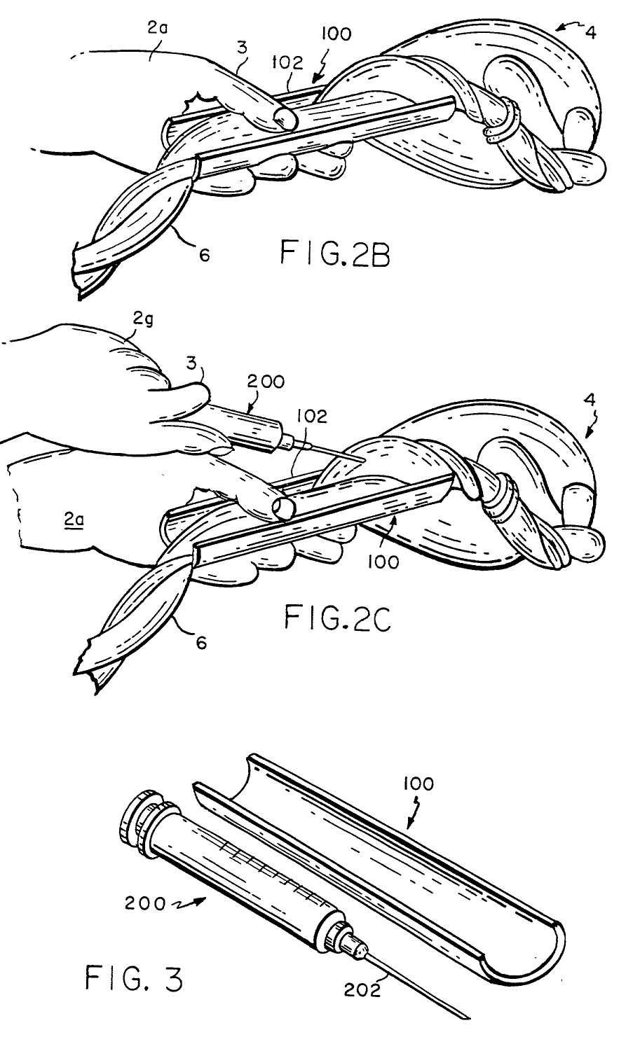

The embodiment of a blood collection shield 100 illustrated in FIGS. 1A-C is generally configured and arranged so as to form a unitary structure including a passage or trough portion 102 and a bottom portion 104. The trough portion 102 is generally configured so as to be capable of releasably receiving therein a body portion, more specifically the umbilical cord 6 (FIG. 2A) extending from the placenta 4 (FIG. 2A). The bottom portion 104 is generally configured and arranged so as to be releasably received in the palm of the hand 2 (FIG. 2A) of the medical technician or practitioner. The side portions 108 of the blood collection shield also are generally configured and arranged such that the hand 2 can securably hold both of the blood collection shield 100 and the umbilical cord 6 disposed in the trough portion 102. In more particular embodiments, the bottom portion 104 and the side portions 108 are configured so that the blood collection shield can be held in the supinated palm of the hand 2. In further embodiments, the surface of the trough portion 102, the bottom portion 104 and/or the sides 108 are configurable with any one of a number of surface artifacts, surface coatings or applied films as is known to those skilled in the art to improve grippability of the blood collection shield 100 by the non-dominant hand 2a and retention of the umbilical cord within the trough portion.

In an illustrative embodiment, the blood collection shield 100 is in the general shape and form of a cylindrical tubular member that is cut about in half along the length of the tubular member. Thus, the through portion 102 and the bottom portion 104 are arcuate in cross-section. This is not a limitation, however, as other geometrical shapes are contemplated. For example, and as illustrated in FIG. 1D, in another embodiment, a blood collection shield 100a is in the general shape and form of a U-shaped member. In yet another embodiment, and as illustrated in FIG. 1E, a blood collection shield 100b is in the general shape and form of a polygonal member.

The blood collection shield 100 is made of any of number of materials known to those skilled in the art that are appropriate for a typical hospital use such as, for example, blood withdrawal, catheter emplacement and other medical procedures utilizing needles. In addition, such materials shall include those that are of the type that would be generally resistant to puncture by a needle under typical hospital use. Such materials include, but are not limited, to stainless steel (e.g., 304 stainless steel), medical grade plastic or similar materials suitable for use in a medical setting. The medical grade plastic is sterilized using any of a number of techniques known to those skilled in the art including for example chemical, gamma radiation and ultraviolet light.

In more specific embodiments, the blood collection shield 100 is reusable and sterilizable between uses. Alternatively, the blood collection shield 100 is disposable and thrown out after each use, thereby eliminating possibility of cross contamination from one patient to another.

The blood collection shield 100 also can be manufactured using any of a number of techniques known to those skilled in the art for the type of material making up the blood collection shield. Such methods include, but are not limited to, molding or stamping the starting material into the final configuration of the blood collection shield. In an exemplary illustrative embodiment, the blood collection shield 100 is made of Grade 304 stainless steel.

In illustrative exemplary embodiments, a blood collection shield 100 for an umbilical cord application is configured and arranged as shown in FIGS. 1A-C. Such a blood collection shield 100 is generally dimensioned so as to have a width in the range of from about 1.25 inches to about 1.5 inches, more particularly 1.375 inches; a length in the range of from about 5 to about 7 inches, more particularly about 6 inches, a height in the range of from about 0.5 to 1.0 inches, more particularly 0.75 inches; and the thickness thereof in the range of from about 0.01 to about 0.1, more particularly about one sixteenth of an inch.

The blood collection methodology according to the present invention, including the use of any of the above-described blood collection shields 100, 100a, 100b of the present invention is best understood from the following discussion taken with reference to FIGS. 2A-C. Reference also should be made to the foregoing discussion and FIGS. 1A-C for details regarding any feature or element referred to hereinafter not otherwise described below. Although the methodology is described in the following in connection with drawing cord blood from the umbilical cord that has been detached from a newborn infant using any of a number of techniques known in the art, the methodology of the present invention is not limited to this use.

Prior to use, the blood collection shield 100 typically is removed from its protective sterile packaging and otherwise prepared for the process of withdrawing blood from the umbilical cord. After unshipping, the medical technician or practitioner takes the blood collection shield 100 and places it in and holds it in the supinated palm of the non-dominant hand 2a. The blood collection shield 100 is more particularly positioned so that the trough portion 102 faces generally away from the palm of the non-dominant hand 2a. See generally FIG. 2A.

Thereafter, the umbilical cord 6 is located in the trough portion 102. More particularly, the umbilical cord is positioned so the cut and clamped end of the umbilical cord extends out the end of the blood collection shield generally toward the heal of the palm and the end of the umbilical cord attached to the placenta 4 extends the end towards the fingers. In addition, the thumb 3 of the non-dominant hand 2a is used to stabilize the umbilical cord 6. In this configuration the non-dominant hand is holding both the blood collection shield 100 and the umbilical cord 6 and establishes a stable secure area for venapuncture. See generally FIG. 2B.

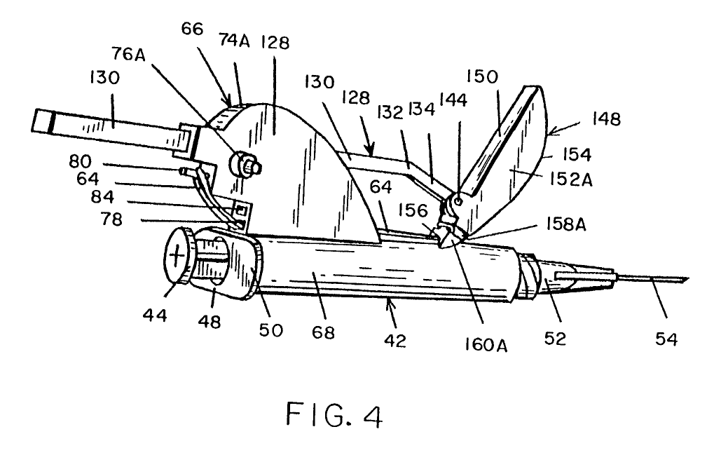

Thereafter, the medical technician/practitioner, using their dominant hand 2b, inserts the needle 202 (FIG. 3) of a syringe 200 into the umbilical cord 6 to withdraw the cord blood for analysis or other use as herein described and known to those skilled in the art. More specifically, the needle 202 is inserted above the thumb 3 of the non-dominant hand 2a. In further embodiments, the blood collection shield 100 is angled in a direction toward the placental basin (not shown) so as to direct any blood escaping into the placental basin. See generally FIG. 2C.

Such a configuration advantageously positions the blood collection shield 100 between the umbilical cord 6 and the non-dominant hand 2a. Thus, the risk of accidental needle sticks of the operator, in particular to the digits or extremities of the non-dominant hand 2a, are minimized or prevented. Correspondingly, the health risk attributable to such accidental needle sticks is minimized as well as minimizing secondary health issues arising while awaiting test results of tests performed after such an accidental stick.

After insertion, the medical technician/practitioner operates the syringe or other blood extraction device or mechanism known in the art, so as to withdraw a quantity of cord blood. After withdrawing the desired amount of cord blood, the needle is extracted from the umbilical cord 6. In further embodiments, the syringe 200 is of a type that protects the needle 202 in such a fashion so as to prevent further accidental needle sticks. There is shown in FIG. 4, and with reference to U.S. Pat. No. 5,152,751, a manually operated needle shielding safety device that is attachable to a standard hypodermic needle and syringe medical apparatus 42. All of the teachings of U.S. Pat. No. 5,152,751, including teachings other than the shielding device disclosed therein, are adopted herein by reference.

For the illustrated safety device, by forward directed thumb or finger stroke upon a proximally projecting pushrod 128, that is slidably operable within a pushrod channel 82 positioned parallel to and radially removed from the primary syringe barrel plunger 44, the elongated shield 148 is pushed from a housing 66 mounted at the base of the hypodermic needle and syringe apparatus 42 and rotated longitudinally from an inverted position. The elongated shield 148 also is thus advanced to the distal end of the syringe apparatus 42 against resistance from a rubber band 64 or integral expansion spring 244. The distal portion of the shield 148 surmounts the tip of the needle 54 and the needle 54 enters the shield 148 laterally through an opening in the shield face 154. The operator then releases thumb or finger pressure upon the pushrod 128 and the enclosed forward end of the shield 148 is pulled rearward by the rubber band 64 or integral expansion spring 244 to surround and safely contain the needle 54 tip.

Such a safety device can be deployed quickly, surely, and conveniently at the user's option immediately after the injection process, with one hand, without the need to shift finger grip upon the hypodermic apparatus 42, and without interfering with the user's concentration upon other important medical tasks. Such a safety device can be used with most currently manufactured needle and syringe devices and does not require modification of existing needle designs, needle hub designs including luer connection designs and low-dose minimum “dead space” designs, needle cementing processes and equipment, fluid chamber designs, and fluid chamber piston engines.

Device kits also are provided that comprise one or more blood collection shields according to the present invention, preferably packaged in a sterile condition. Kits of the invention also may include one or more needle and syringes as is known in the art for use with the blood collection shield, preferably packaged in sterile condition, and/or including written instructions for the collection of blood such as cord blood and other components of the kit. Such syringes include those having or adapted for use with needle shields to prevent further needle sticks, such as the mechanism illustrated in FIG. 4. There is shown in FIG. 3 a blood collection shield 100 and a syringe 200 that illustratively comprise a device kit according to the present invention.

Although a preferred embodiment of the invention has been described using specific terms, such description is for illustrative purposes only, and it is to be understood that changes and variations may be made without departing from the spirit or scope of the following claims.

What is claimed is:

1. A method for collection of blood comprising:
positioning a body portion, from which blood is to be withdrawn, within a shield member such that the shield member is generally disposed between the body portion and a hand of a user; and
inserting an insertion member of a blood extraction device into the body portion such that the shield member is generally disposed between the insertion member and the user's hand.

2. The method of claim 1 further comprising holding the shield member in one hand of the user while accomplishing said inserting using another hand of the user.

3. The method of claim 1 further comprising stabilizing the body portion while holding the shield member.

4. The method of claim 3 wherein said stabilizing includes stabilizing the body portion using a digit of the user's hand.

5. The method of claim 1 further comprising providing a shield member, one portion of which is configured to be releasably held in said one hand and another portion of which is configured to releasably receive the body portion.

6. The method of claim 5 wherein the another portion of the provided shield member includes a surface recess extending along an axis of the shield member, in which is received the body portion.

7. The method of claim 6 wherein the recess and said one portion of the provided shield member is each one of arcuate or polygonal in cross-section.

8. The method of claim 7 wherein the provided shield member is configured to be in the general shape of a hollow tube cut in half along its length.

9. The method of claim 1, further comprising withdrawing blood from the body portion using the blood extraction device following said step of inserting.

10. The method of claim 9 further comprising:
stopping the withdrawal of blood; and
withdrawing the insertion member of the blood extraction device from the body portion.

11. The method of claim 4 further comprising:
stopping the withdrawal of blood;
withdrawing the insertion member of the blood extraction device from the body portion;
removing the digit of the user's hand; and
removing the body portion from the shield member.

12. The method of claim 1 wherein the blood is umbilical cord blood.

13. The method of claim 1 wherein the body portion is the umbilical cord, wherein the inserting includes inserting the insertion member into the umbilical cord.

14. The method of claim 13 further comprising withdrawing cord blood from the umbilical cord using the blood extraction device following the step of inserting the insertion member.

15. The method of claim 14 wherein the blood extraction device comprises a needle and syringe.

16. A method for collection of umbilical cord blood comprising:
positioning an umbilical cord within a shield member; and
contacting the cord with a blood extraction device.

17. The method of claim 16 wherein the shield member is generally disposed between the cord and a hand of a medical practitioner.

18. The method of claim 16 wherein the blood extraction device comprises a needle and syringe.

19. A shielding device comprising:
a cradle member, the cradle member being configured so one portion thereof is releasably held in a hand of a user, so another portion thereof releasably receives the body portion, and so a digit of the user's hand secures the body portion in the another portion while holding the cradle member.

20. The shield device of claim 19 wherein said another portion includes a surface recess extending along an axis of the cradle member, in which recess is received the body portion.

21. The shield device of claim 19 wherein the recess is one of arcuate or polygonal in cross-section.

22. The shield device of claim 20 wherein the one portion is configured so as to be one of arcuate or polygonal in cross-section.

23. The shield device of claim 19 wherein the cradle member is configured and arranged so as to be in the general shape of a hollow tube cut in half along its length.

24. The shield device of claim 19 wherein the cradle member is made from stainless steel and/or medical grade plastic.

25. An apparatus for extracting cord blood from an umbilical cord comprising:
a cradle member, said cradle member being configured and arranged so one portion thereof is releasably held in a hand of a user, so another portion thereof releasably receives the umbilical cord, and so a digit of the user's hand secures and stabilizes the umbilical cord in said another portion while holding the cradle member; and
a blood extraction device including an insertion member being configured so as to inserted into the umbilical cord and to withdraw cord blood therefrom.

26. A blood collection device kit comprising:
a shielding device including a cradle member, said cradle member being configured and arranged so one portion thereof is releasably held in a hand of a user, so another portion thereof releasably receives the body portion, and so a digit of the user's hand secures and stabilizes the body portion in said another portion while holding the cradle member.

27. The blood collection kit of claim 26 further comprising
a blood extraction device including an insertion member that is configured to be inserted into the body portion and to withdraw blood therefrom.

28. The blood collection kit of claim 26 wherein the cradle member is configured and arranged so as to be in the general shape of a hollow tube cut in half along its length and the blood extraction device comprises a needle and syringe.

29. The blood collection kit of claim 26 wherein the cradle member is configured and arranged so as to be in the general shape of a hollow tube cut in half along its length.

30. The blood collection kit of claim 26, wherein said another portion includes a surface recess extending along an axis of the cradle member, in which recess is received the body portion and wherein the recess is one of arcuate or polygonal in cross-section.

31. The blood collection kit of claim 26 wherein the body portion is an umbilical cord and wherein the recess is configured and arranged to receive the umbilical cord therein.

32. The blood collection kit of claim 27 wherein the blood extraction device comprises a needle and syringe.

33. The method of claim 10, further comprising the step of covering at least an end of the insertion member following said withdrawing the insertion member from the body portion.

34. A method for collection of blood from an umbilical cord (cord blood), comprising:
providing a shield member, one portion of which is configured to be releasably held in one hand of a user and another portion of which is configured to releasably receive the umbilical cord, wherein the another portion includes a surface recess extending along an axis of the shield member, in which is received the umbilical cord and wherein the recess and the one portion are each arcuate in cross-section;
holding a shield member with one hand of a user;
positioning the umbilical cord within the shield member such that the shield member is generally disposed between the umbilical cord and the one hand;
stabilizing the umbilical cord while holding the shield member using the one hand;
inserting an insertion member of a blood extraction device into the umbilical cord using another hand of the user such that the shield member is generally disposed between the insertion member and the one hand; and
withdrawing the cord blood from the umbilical using the blood extraction device;

35. The method of claim 34 further comprising:
stopping the withdrawal of cord blood; and
withdrawing the insertion member from the umbilical cord.

36. A shielding device comprising:
a cradle member, the cradle member being configured so one portion thereof is releasably held in a hand of a user, so another portion thereof releasably receives the body portion, and so a digit of the user's hand secures the body portion in the another portion while holding the cradle member;
wherein said another portion includes a surface recess extending along an axis of the cradle member, in which recess is received the body portion; and
wherein the recess and said one portion are configured so as to be arcuate in cross-section.

37. A cord blood collection device kit comprising:
a shielding device including a cradle member, the cradle member being configured and arranged so one portion thereof is releasably held in a hand of a user, so another portion thereof releasably receives an umbilical cord, and so a digit of the user's hand secures and stabilizes the umbilical cord in said another portion while holding the cradle member;
a needle and syringe to withdraw cord blood from the umbilical cord, the needle being configured to be inserted into the umbilical cord; and
wherein said another portion includes a surface recess extending along an axis of the cradle member that is configured and arranged to receive the umbilical cord therein and wherein the recess is arcuate in cross-section.
